(19) United States
(12) Patent Application Publication
(10) Pub. No.: US 2005/0096559 A1
(43) Pub. Date: May 5, 2005

(54) Sensor sheet

(75) Inventor: Kenichi Yanai, Nisshin-city (JP)

Correspondence Address: POSZ LAW GROUP, PLC, 11250 ROGER BACON DRIVE, SUITE 10, RESTON, VA, 20190, US

(73) Assignee: DENSO CORPORATION

(21) Appl. No.: 10/944,713

(22) Filed: Sep. 21, 2004

(30) Foreign Application Priority Data
Oct. 29, 2003 (JP) 2003-369366

(51) Int. Cl. A61B 5/08; A61B 5/103

(52) U.S. Cl. 600/534; 600/595

(57) ABSTRACT

A sensor sheet has a substantially rectangular sheet member and a plurality of sensing elements disposed in a predetermined distribution on the sheet member and sense pressure or vibration. The sensor sheet outputting a signal corresponding to load or vibration from a sleeper. A lateral width of the sheet member is set to a minimum length at which the sensing elements exist in a predetermined range when the sleeper moves in a lateral direction. A longitudinal length of the sheet member is set to a minimum length at which the sensing elements exist in a predetermined range corresponding to a trunk part of the sleeper. The sheet member is allowed to be folded at least in the lateral width direction.

### CROSS REFERENCE TO RELATED APPLICATION

This application is based upon, claims the benefit of priority of, and incorporates by reference the contents of Japanese Patent Application No. 2003-369366 filed on Oct. 29, 2003.

### FIELD OF THE INVENTION

The present invention relates to a sensor sheet used to detect living body information.

### BACKGROUND OF THE INVENTION

JP-A-2001-37742 (Patent Document 1) discloses a device for measuring an apnea state or low aspiration state of a sleeper due to for example, sleep apnea syndrome. This device is conventionally equipped with a sensor sheet which is inserted to the lower portion of bedding and has plural sensing elements, a controller, a monitor for displaying the respiratory rate, reduction frequency of oxygen saturation degree in the blood, etc. of a sleeper, etc.

The sensor sheet contains pressure-sensitive elements which vary in electrical resistance in accordance with applied weight and are arranged at equal intervals on plural sheet members, and it is inserted into the lower portion of a bedding such as mattress or the like put on a bed. The controller generates a respiration body movement signal which is a signal component of a frequency band corresponding to the respiratory rate of a sleeper on the basis of the load signal output from each pressure-sensitive element. The decrease of the oxygen saturation degree in blood which occurs at an obstructive apnea time is judged on the basis of a variation pattern in amplitude of the respiration body movement signal, and the frequency of the decrease of the oxygen saturation degree in blood is displayed on a monitor.

However, in the conventional device as described above, the sensor sheet is disposed on the overall surface of the bed. Thus, it is basically adapted to a case where the bed concerned is fixedly equipped as a bed for examination as described above. Such a sensor sheet is inconvenient because it requires that a person being examined (such as a patient) visit a hospital or the like in which such a bed is set up every time the person is examined. It is more preferable that the sensor sheet be more portable so that a person being examined be able to bring the sensor sheet back to his/her home and be examined while sleeping on his/her bed or the like. One such portable device has been disclosed in which various sensors are attached to a human body. However, this device is also inconvenient because of the extensive time required to set it up.

In a conventional sensor sheet type a user merely lies in bed. The sensor sheet is placed on the overall surface of the bed and thus it is designed as a large-size sheet. As a result, it is not easy to bring a set of examining device containing such a sensor sheet from a hospital or the like back to home.

Further, if the device is made more compact, the detection precision will become lower.

Therefore, the present invention has been implemented in view of the foregoing problem, and has an object to provide a sensor sheet which implements portability and maintains detection precision.

### SUMMARY OF THE INVENTION

In order to attain the above object, a sensor sheet according to the present invention is used in a device for detecting living body information such as respiration information or the like, for example, and it has plural sensing elements which are arranged in a predetermined distribution on a substantially rectangular sheet member and sense pressure or vibration. The lateral width of the sheet member is set to the minimum length at which the sensing elements exist in a predetermined range when the sleeper moves in a lateral direction due to roll-over or the like in his/her sleep, and the longitudinal length of the sheet member is set to the minimum length at which the sensing elements exist in a predetermined range corresponding to a trunk part of the sleeper.

The conventional sensor sheet is adapted to a case where it is fixedly set up as an examining bed because it is disposed on the overall surface of a bed. However, the sensor sheet of the present invention is designed to have a dimension as described above and also can be folded. Therefore, it is compact and also enhanced in portability, so that a person being examined can easily bring it back to his/her home for use. A type of conventional device that a person being examined brings back to his/her home to carry out an examination while lying in bed or the like at home is carried out while various kinds of sensors are attached to the human body, however, this device needs much time for the setting thereof and thus is inconvenient. On the other hand, in the case of the present invention, it is merely disposed below a sleeper, and thus the setting thereof is very simple.

The sensing elements are arranged so as to exist in a predetermined range corresponding to the trunk part of the person being examined which is required to detect respiration information, etc. and in a predetermined range when the person being examined moves in the lateral direction due to roll-over in his/her sleep, and thus the detection precision is also high.

With respect to the lateral width of the sheet member, if it is assumed that the sheet member is disposed on a semi-double bed, it is sufficient that the lateral width is equal to the bed width of 1200 mm so that it can adapt to a single bed.

On the other hand, with respect to the longitudinal length, the following devices may be considered in consideration of a detection target, precision, etc.

(1) to (3) are described in decreasing order of size.

(1) The longitudinal length is determined on the basis of the interval between tibial height and crotch height of the sleeper.

This is the length corresponding to “the overall trunk part”, and if it is assumed that respiration information is detected as living body information, the center of gravity of the respiratory movement is slightly displaced in the longitudinal direction by the respiratory movement itself. Accordingly, frequencies caused by respiration strongly appear with different phases under the chest part and under the backside. All the load variations caused by respiration can be detected by grasping the overall range in which the respiratory frequencies appear, and such a size as described above is preferable for the above purpose. For example, in the case of an average Japanese adult, the longitudinal length corresponds to about 800 mm.

(2) The longitudinal length is defined on the basis of the interval between tibial height and the sidebone.

This is the length corresponding to “upper trunk part”. When it is assumed that respiration information detected as living body information, a detection result would be achieved with sufficient precision if respiration information on only the upper trunk part is achieved. For example, in the case of an average Japanese adult, it corresponds to the length of about 450 mm.

(3) The longitudinal length is defined on the basis of the length of the chest part in the body height direction of the sleeper.

This is the length corresponding to “chest part”, and if it is assumed that respiration information is detected as living body information, the detection only in the above range would be sufficient when only the chest part respiration frequency is detected. For example, in the case of an average Japanese adult, it corresponds to the length of about 60 mm.

In the case of the length of about 60 mm, the sensor sheet is very compact as a whole when it is folded in the lateral direction, and thus portability is very enhanced. Of course, this is applied to only the case where only the chest part respiration frequency is detected, however, this is very effective to a person being examined (patient) to which the detection as described above is sufficient. In the case of the size of (1) which is the maximum size, it is equal to about 800 mm, and this is a easily portable level.

The construction which can be folded will be described. The folding may be a two-fold type or three-fold or more type. In this case, it is required that no sensing element exists at mountain (or valley) portions when folded. Such an arrangement itself can be easily implemented, however, the following points must be considered when a construction that transmission pieces disposed at one surface side of a sheet member so as to project from the surface side transmit pressure or vibration to sensor portions arranged in the sheet member is adopted. That is, when the sheet member is folded so that faces from which the transmission pieces project are confronted to each other, the precision of the sensor portions would be lowered if the state that the transmission pieces abut against one another is continued. For example, when the sensor portion are formed of film resin, creep occurs and the precision thereof is lowered in consideration of a situation that it is used and stocked for a long term, which affects a durability characteristic as a sensing element. Therefore, when the sheet member is folded so that the transmission pieces are confronted to each other, they may be disposed in a staggered arrangement with a basis of a sensing element or a sensing element array comprising a plurality of sensing elements so that the transmission pieces do not abut against one another.

In order to prevent the transmission pieces from abutting against one another, the sensing elements may be disposed in a staggered arrangement with a basis of a sensing element or a sensing element array comprising plural sensing elements. However, if the sensing element is disposed in a staggered arrangement with a basis of a sensing element, the sensing elements can be uniformly arranged on the sheet member. Therefore, such a uniform arrangement is expected to be favorable to properly detect living body information.

Such a sensor sheet is used while put on bedding such as a bed or the like, and some dispersion of variation of pressure or vibration of the sensing elements may occur in accordance with the state of the bedding. That is, in the case of flexible bedding, the weight of a sleeper excessively moves the sensing elements at the weighted portion downwardly and thus the sensing elements cannot output the detection signals corresponding to a true load or vibration. Therefore, it is considered that the dispersion in the downward displacement of the sensing elements due to the flexibility of the bedding is suppressed by a hard sheet portion to thereby reduce the dispersion of the pressure or vibration variation of the sensing elements. In order to completely prevent the downward displacement of the sensing elements, a hard sheet portion having very high rigidity may be adopted. However, this makes a sleeper feel uncomfortable to sleep and may prevent the sleeper from sleeping. Therefore, it is preferable to adopt a hard sheet portion considering the comfortableness under sleep insofar as the dispersion of the pressure or vibration variation of the sensing elements can be reduced to the extent that it is permitted. If such a hard sheet portion is disposed on the overall surface of the sheet member, the sheet member cannot be folded, and thus the hard sheet portion may be divided at the folding portion.

With respect to the sheet member, it may be considered that a film projecting portion is disposed on the upper and lower surfaces of the sheet member and the sensing elements are disposed between the film protecting portions to be isolated from the external atmosphere. This construction can prevent the detection performance of the sensing elements from being affected by inability of control of urine flow, sweat, etc. of a sleeper, and thus it is also effective in durability.

If the film protecting portions are designed to be transparent, the internal construction can be easily viewed, and when breaking of wire or other disadvantages occur, the cause therefore can be easily found out. The transparency means “clear and colorless” or “clear and colored”.

When a site needing maintenance (for example, a control box or the like) is provided in the film protecting portion, it is preferable to provide a maintenance hole which can be opened and closed for convenience when the site is maintained.

### BRIEF DESCRIPTION OF THE DRAWINGS

The above and other objects, features and advantages of the present invention will become more apparent from the following detailed description made with reference to the accompanying drawings. In the drawings:

FIG. 1 is a diagram showing the schematic construction of a living body information detecting device having a sensor sheet according to an embodiment;

FIG. 2A is an view of FIG. 1 along line IIA-IIA, FIG. 2B is a diagram when a sensor sheet 2 is folded;

FIG. 3 is a block diagram showing the circuit construction of a controller 3 of the living body information detecting device 1;

FIG. 4 is a diagram showing a state that the living body information detecting device 1 is put on a bed;

FIG. 5 is a diagram showing power spectra of respiration frequencies corresponding to body sites of a sleeper;

FIG. 6 is a diagram showing a result calculated from an area in chest part length in which a respiration frequency component is larger than other frequency components by 10 dB or more; and

FIGS. 7A-7B are diagrams when pressure-sensitive elements are disposed in a staggered arrangement with an array unit.

### DETAILED DESCRIPTION OF THE PREFERRED EMBODIMENTS

Embodiments to which the present invention is applied will be described with reference to drawings. The embodiments of the present invention are not limited to the following embodiments, and various other embodiments may be used insofar as they belong to the technical scope of the present invention.

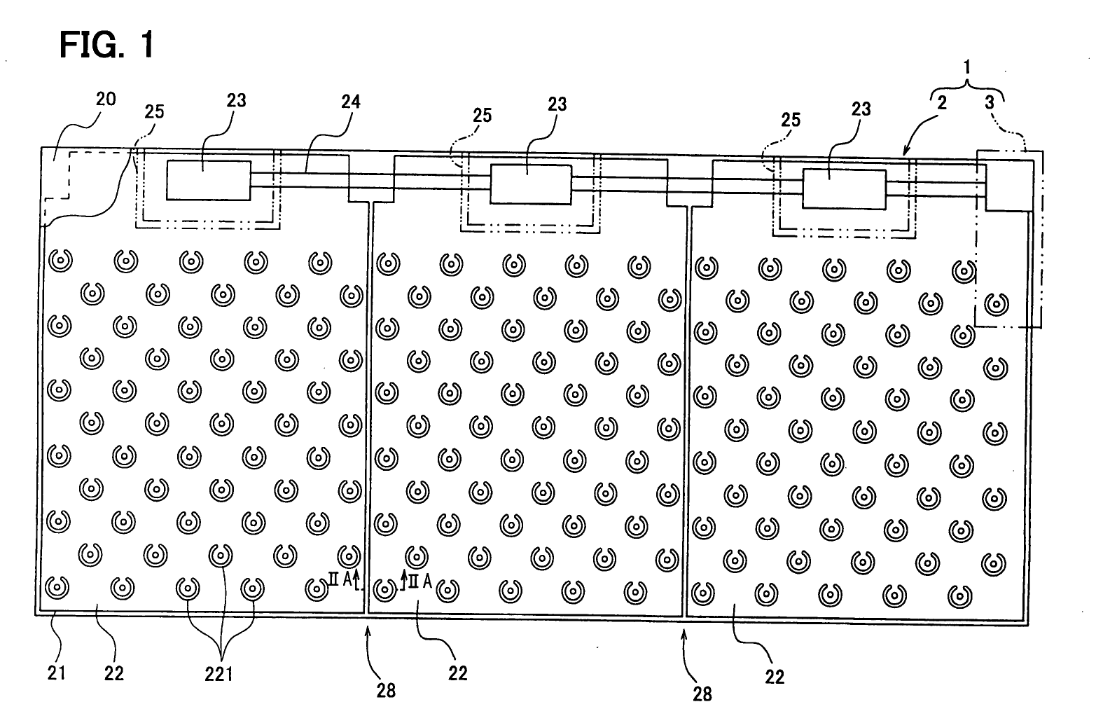

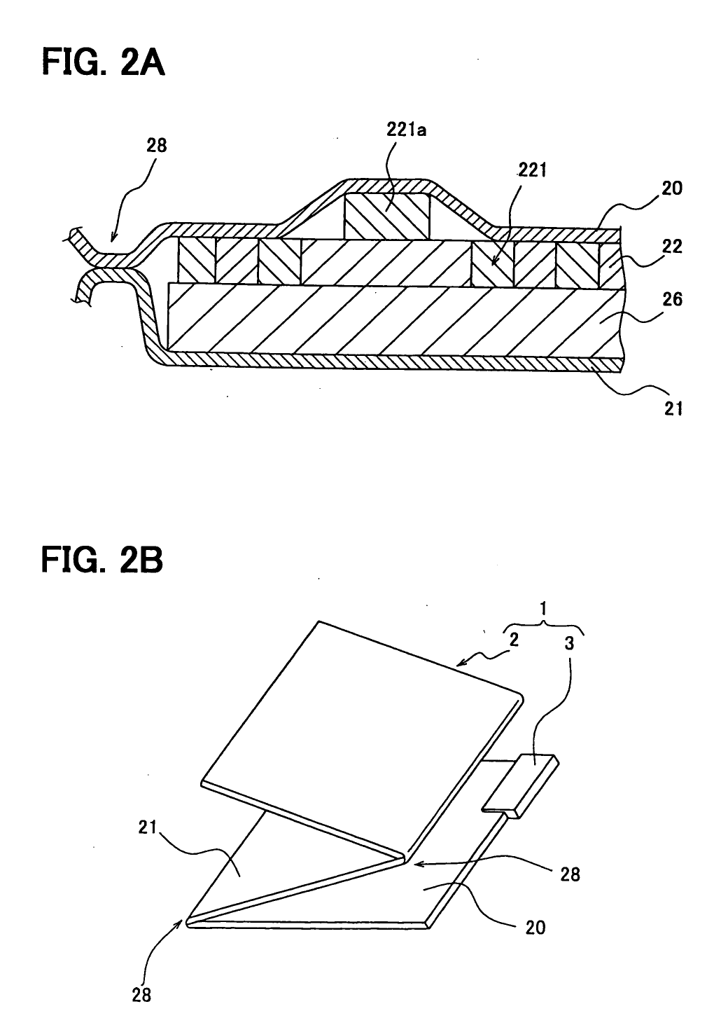

FIG. 1 is a diagram showing the schematic construction of a living body information detecting device 1 having a sensor sheet 2 according to an embodiment, and FIG. 2A is a cross-sectional view (line IIA-IIA of FIG. 1) of a part of the living body information detecting device 1.

As shown in FIG. 1, the living body information detecting device 1 is equipped with a sensor sheet 2 and a controller 3, and the controller 3 is fixed to the end portion of the rectangular sensor sheet 2 (in this embodiment, the right shoulder portion when viewed from the front side). The living body information detecting device 1 of this embodiment is used while laid out on a bed 50 as shown in FIG. 4, for example. The bed 50 comprises a support portion 51 on which lying bedding 60 such as a mattress or the like is put, and a back board portion 52 equipped so as to stand from the end portion of the support portion 51, and the living body information detecting device 1 is used while inserted to the lower portion of the lying bedding 60 put on the support portion 51 of the bed 50.

The living body information detecting device 1 is disposed to be nearer to the back board portion 52 side than the center portion of the support portion 51 so that it faces the trunk part of a sleeper when the sleeper lays on the bed 50.

The sensor sheet 2 will be first described with reference to FIG. 1 and FIGS. 2A-2B. The sensor sheet 2 comprises plural layers as shown in the sectional view of FIG. 2A, and an upper PU film 20, a pressure-sensitive element layer 22, a PVC sheet 26 and a lower PU film 21 are laminated from the upper side in this order.

The upper PU film 20 and the lower PU film 21 are formed of soft and transparent polyurethane resin film, and correspond to the “film protecting portion”. The upper PU film 20 and the lower PU film 21 are designed to have the same rectangular shape as the overall shape of the sensor sheet 2, and the respective four sides thereof are connected to one another. As a result, the pressure-sensitive element layer 22, etc. disposed in the film protecting portions are isolated from the external atmosphere.

The pressure-sensitive element layer 22 is divided into three equal parts in the longitudinal direction of the rectangular sensor sheet 2. The respective pressure-sensitive element layers 22 thus tri-sectioned have the same construction, and fifty five pressure-sensitive elements 221 as “sensing elements” whose electrical resistance varies (decreases) in accordance with an applied load are regularly arranged on each of the pressure-sensitive element layers 22. Specifically, an array of six aligned pressure-sensitive elements and an array of five aligned pressure-sensitive elements are alternately arranged in the direction perpendicular to the longitudinal direction of the sensor sheet 2 so that totally 10 arrays are arranged, and the pressure-sensitive elements 22 of the respective neighboring arrays are disposed in a staggered arrangement. The arrangement of the photosensitive elements 221 is identical among the respective pressure-sensitive element layers 22, and at the boundary portion between the tri-sectioned pressure-sensitive element layers 22, an array of six aligned pressure-sensitive elements 221 of one pressure-sensitive element layer 22 and an array of five aligned pressure-sensitive elements 221 of the other pressure-sensitive element layer 22 are adjacent to each other, and thus the pressure-sensitive elements 221 of these arrays are disposed in a staggered arrangement. As shown in FIG. 2A, rubber pressure pieces 221a as “transmission pieces” are fixed to the upper surfaces of the respective pressure-sensitive elements 221 with sticky agent, adhesive agent or the like.

In pressure-sensitive element layer 22, when the sensor sheet 2 is set on the support portion 51 of the bed 50, no pressure-sensitive element 221 is disposed in a predetermined range located at the back board portion 52 side, and sensor selecting portions 23 are disposed in this area. The sensor selecting portions 23 arranged on the three pressure-sensitive element layers 22 are connected to one another by a film type wire 24, and the sensor selecting portion 23 at the rightmost side in FIG. 1 is connected to the controller 3. Furthermore, in FIG. 1, a wire pattern for electrically connecting the respective pressure-sensitive elements 221 and the sensor selecting portions 23 is omitted from the illustration. When a voltage is applied to a circuit containing each pressure-sensitive element 221, the electrical resistance of the pressure-sensitive element 221 is varied in accordance with an applied load, so that a drop voltage value caused by the pressure-sensitive element 221 is increased/reduced. Therefore, the applied load can be detected independently for every pressure-sensitive element 221 on the basis of the variation of the drop voltage value.

The upper PU film 20 is equipped with a maintenance hole 25 which can be opened/closed at each site at which each sensor selecting portion 23 is disposed. Specifically, the maintenance hole 25 which is slightly larger than each sensor selecting portion 23 is formed, and the upper PU film 20 which is slightly larger than the maintenance hole 25 is covered on the maintenance hole 25 so that the maintenance hole 25 can be opened and closed. This construction enhances the convenience when the sensor selecting portions 23, the film type wire 24 for connecting these, etc. are maintained.

The PVC sheet 26 is formed of hard vinyl chloride resin sheet, and corresponds to the “hard sheet portion”. The PVC sheet 26 has the same shape as the pressure-sensitive element layer 22, and it is disposed while divided into three equal parts in the longitudinal direction of the rectangular sensor sheet 2. With respect hardness of the PVC sheet 26, it is preferable to consider the following points. That is, the sensor sheet 2 is used while laid on the bed 50 or the like as described above, however, some dispersion may occur in pressure variation of the pressure-sensitive elements 221 in accordance with the state of the bedding. That is, in the case of flexible bedding, the weight of the sleeper causes the pressure-sensitive elements at that portion to be excessively displaced downwardly, and thus it is impossible to output the pressure signal corresponding to the true applied weight. Therefore, the downward displacement of the pressure-sensitive elements 221 due to the flexibility of the bedding is suppressed by the PVC sheet 26, and the dispersion in pressure variation of the pressure-sensitive elements 221 is reduced. In order to completely eliminate the downward displacement of the pressure-sensitive elements 221, a PVC sheet 26 having very high rigidity may be used. However, this sheet makes a sleeper feel uncomfortable to sleep and it may disturb sleeping itself. Therefore, the sheet is preferably designed to be suitably hard in consideration of comfortableness to sleep insofar as the dispersion of the pressure variation of the pressure-sensitive elements 221 is allowable.

In the foregoing embodiment, polyurethane resin film is used for the upper PU film 20 and the lower PU film 21, and vinyl chloride resin sheet is used as the PVC sheet 26. However, the present invention is not limited to these materials, and other resin film or resin sheets may be used. Furthermore, a film or sheet which is not formed of resin may be used.

The upper PU film 20, the pressure-sensitive element layer 22, the PVC sheet 26 and the lower PU film 21 are constructed as described above, so that no pressure-sensitive element layer 22 and no PVC sheet 26 exist at the folding portions 28 shown in FIG. 8, and only the upper PU film 20 and the lower PU film 21 exist there. As shown in FIG. 2A, the upper PU film 20 and the lower PU film 21 are joined to each other, and the sensor sheet 2 can be folded at the folding portions 28. The film type wire 24 partially exits, however, the film type wire 24 is designed to have high durability to the folding, so that no trouble occurs at the folding time. The folding portion 28 at the left side in FIG. 1 is valley-folded, and the folding portion 28 at the left side is mountain-folded, so that the sensor sheet can be three-folded as shown in FIG. 2B. When the sensor sheet is folded as described above, the faces on which the upper PU film 20 are equipped abut against each other. However, the pressure-sensitive elements 221 are disposed in the staggered arrangement as shown in FIG. 1, so that the rubber pressure pieces 221a are prevented from abutting against (interfere with) one another when folded.

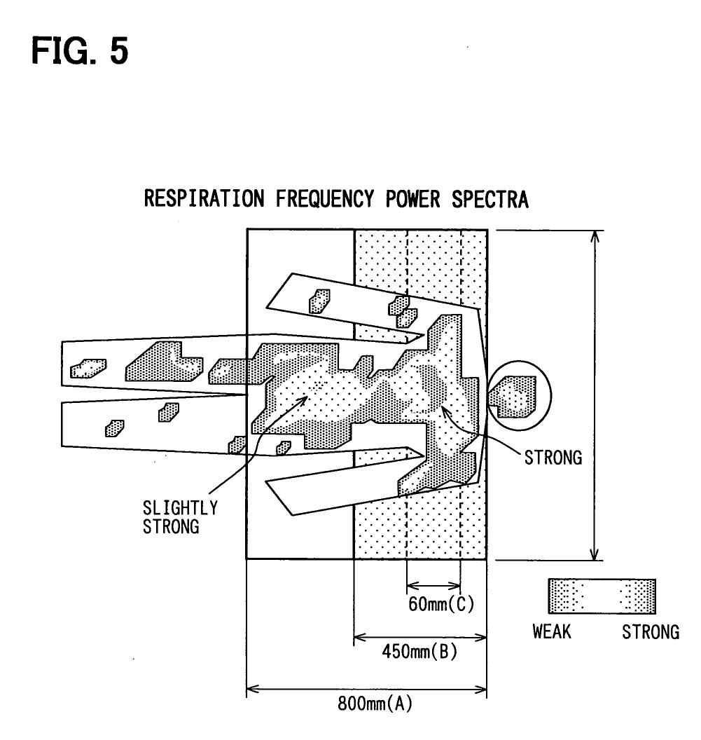

Here, the size of the sensor sheet 2 will be further described. In this embodiment, the lateral width is matched with the width of the bed 50, however, some variations may be considered for the longitudinal length in consideration of the detection target, the precision, etc. FIG. 5 shows respiration frequency power spectra corresponding to the human body sites of a sleeper. It is originally a color image, but describing schematically, colored portions in the shape of the human body correspond to sites at which power spectra having a predetermined level or more can be achieved. Of these sites, a strong power spectrum is achieved around the chest, a slightly strong power spectrum is achieved around the center of the waist and weak power spectra are achieved at the other sites. The following three kinds of variations will be described in consideration of an occurrence distribution and an intensity distribution of the respiration frequency power spectra as described above.

(A) The size is defined on the basis of the interval between tibial height and crotch height of the sleeper.

Referring to FIG. 5, if the measurement is carried out in the range between tibial height and the crotch height, substantially the overall range in which the respiration frequencies appear can be grasped, and thus all the load variation caused by respiration can be detected. For example, in the case of an average Japanese adult, it corresponds to the length of about 800 mm. If the bed is assumed as a semi-double bed, the width thereof is equal to about 1200 mm. Therefore, if the sensor sheet is three-folded, the size of each part is equal to 400 mm×800 mm, and this is a sufficiently portable size.

(B) The size is defined on the basis of the interval between tibial height and the sidebone.

Referring to FIG. 5, it is considered that sufficient precision respiration information can be achieved without any information of the waist portion, and thus respiration information on so-called “upper chest part” is detected to carry out size-down. For example, in the case of an average Japanese adult, it corresponds to the length of about 450 mm. Therefore, if the sensor sheet is three-folded, the size of each part is equal to 400 mm×450 mm, so that the sheet can be very easily carried.

(C) The size is defined on the basis of the length of the chest part in the body height direction of the sleeper.

Referring to FIG. 5, a strong spectrum is achieved at the chest portion, and thus if the information on this portion can be detected at minimum, the respiration information can be achieved with some degree of precision. Accordingly, when the size-down (further cost down) is further required, such a size is allowed. For example, in the case of an average Japanese adult, it corresponds to the length of about 60 mm. Therefore, if the sensor sheet is three-folded, the size of each part is equal to 400 mm×60 mm. This means that the sensor sheet can be readily accommodated in a bag or the like, so that the sensor sheet can be further easily carried.

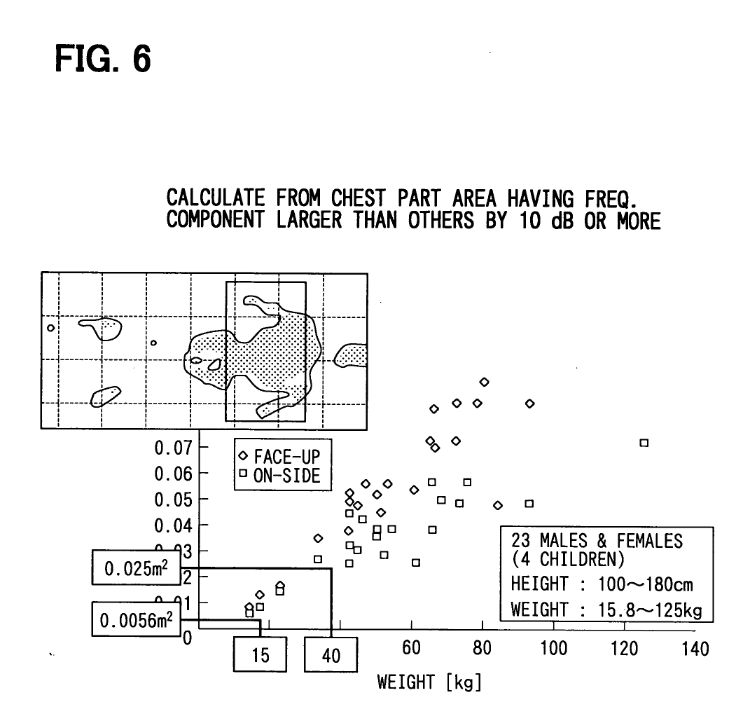

With respect to the interval (pitch) when the pressure-sensitive elements 221 are arranged, the following devices may be considered in view of the following point. FIG. 6 shows a calculation result achieved on the basis of an area in chest part length in which the respiration frequency component is larger in magnitude than the other frequency components by 10 dB or more. Details of an experiment is omitted, the following result was achieved from this experiment as the relation between the weight and the minimum area in which the respiration frequency appears:
0.0056 m² in the case of an infant of 15 kilograms in weight
0.025 m² in the case of an adult of 40 kilograms in weight

Accordingly, in order to adapt to such an area, the arrangement pitch may be set to 7 cm or less for infants and 15 cm or less for adults. Sensor sheets 2 having arrangement pitches exclusive for infants and adults may be prepared. However, it may be adopted that a sensor sheet 2 having a minute arrangement pitch for infants and every other pressure-sensitive elements 221 are validated and used when it is used for adults. With this construction, it is unnecessary to prepare for different sensor sheets 2 for infants and adults, respectively.

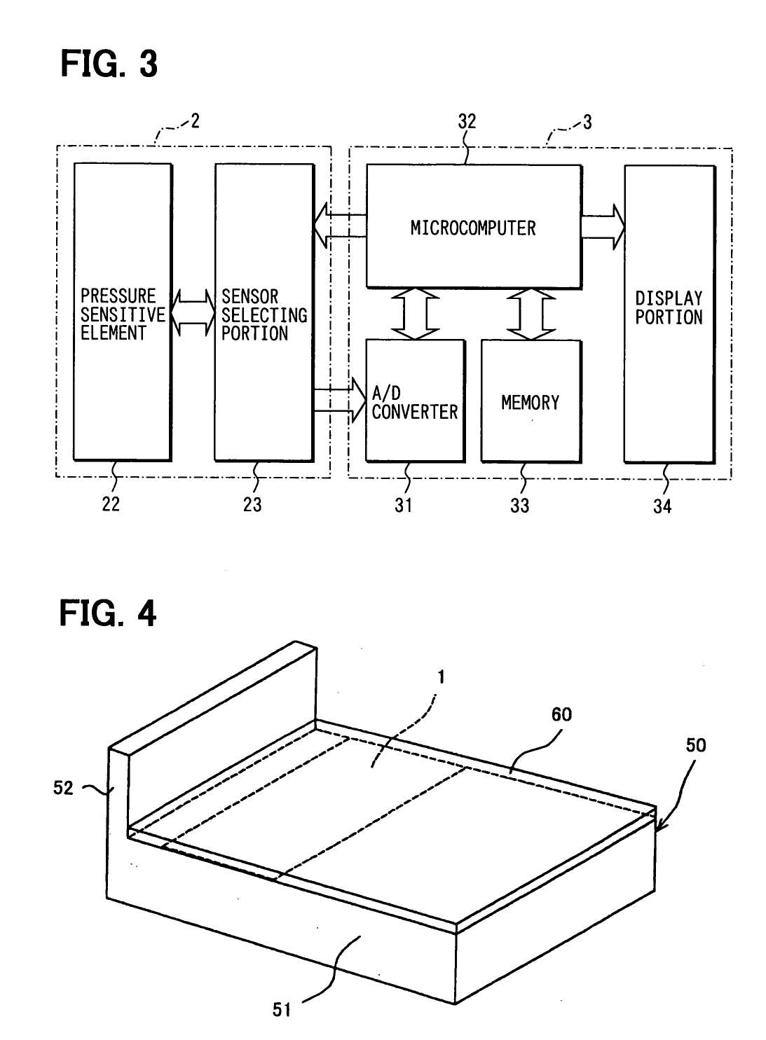

Next, the controller 3 will be described. As shown in the block diagram of FIG. 3, the controller 3 comprises an A/D converter 31, a microcomputer 32, a memory 33 and a display portion 34. In the controller 3, the load signals of the respective pressure-sensitive elements 221 of the pressure-sensitive layer 22 are successively selected by the sensor selecting portion 23, and the values (hereinafter referred to as AD values) achieved by converting the load signals corresponding to analog values to digital values by the A/D converter 31 are taken into the microcomputer 32. At this time, the microcomputer 32 supplies a switching signal to the sensor selecting portion 23 to switch a load signal to be input. By repeating the operation described above, the microcomputer 32 takes in the load signals from all the press-sensitive elements 221 periodically, and stores them into the memory 33.

On the basis of the load signals thus stored, the microcomputer 32 carries out the processing according to the processing based on a predetermined processing program to generate a respiration curve (respiration signal), and also outputs to the display portion 34 of an occurrence frequency, time, etc. of an apnea stare or a low respiration state judged on the basis of the respiration curve. In the display portion 34, the respiration curve and the occurrence frequency, the time, etc. of the apnea state or the low respiration state are displayed in the form of numerical values or graph.

(1) The sensor sheet 2 of this embodiment may be designed to have a longitudinal length of 800 mm even in the case of (A) in [Concerning Size of Sensor Sheet 2], and when it is three-folded, it is reduced to 400 mm×800 mm in size, which is sufficient in size to be easily portable.

The conventional sensor sheet is disposed on the overall surface of a bed, and thus it is adapted to such a case that the bed is fixedly equipped as an examining bed. However, in the case of this embodiment, the sensor sheet is compact and more portable, so that a person being examined can easily bring it back to home for use. With respect to such a conventional device that a person being examined brings a sensor sheet back to his/her home and examined while lying in bed at home, it is used while various kinds of sensors are attached to the human body. However, the setting of this device needs much time, which is inconvenient to the user. On the other hand, in the case of the sensor sheet 2 of this embodiment, it is merely put below a sleeper, and thus the setting is very simple.

Furthermore, the sensing elements are arranged so as to exist in the predetermined range corresponding to the chest part required to detect the respiration information, etc. and in the predetermined range when a sleeper moves in a lateral direction due to roll-over or the like in his/her sleep, and thus the detection precision is also excellent.

(2) Since the pressure-sensitive elements 221 are disposed in a staggered arrangement, the rubber pressure pieces 221a are prevented from abutting against (interfering with) one another when folded. When the state that the rubber pressure pieces 221a abut against one another is continued, the durability characteristic of the pressure-sensitive elements 221 is affected, and thus the sensor precision is lowered. However, such an arrangement can prevent the interference of the rubber pressure pieces 221a, and enhance the durability characteristic.

(3) The internal pressure-sensitive element layer 22, etc. are isolated from the external atmosphere by the upper PU film 20 and the lower PU film 21, so that the detection performance of the pressure-sensitive elements can be prevented from being affected by inability of control of ruin urine flow, sweat, etc. of a sleeper, and thus it is also effective in durability.

Furthermore, since the upper PU film 20 and the lower PU film 21 are transparent, the internal constructions of the pressure-sensitive elements 221, the rubber pressure pieces 221a, the sensor selecting portions 23, the film type wire 24, etc. can be easily viewed, and when breaking of wire or other disadvantages occur, the cause therefore can be easily found out.

(a) In the above embodiment, the sensor sheet is three-folded, however, it may be two-folded or four or more folded. The pressure-sensitive element layer 22 and the PVC sheet 26 are divided into parts whose number corresponds to the folding number, and arranged.

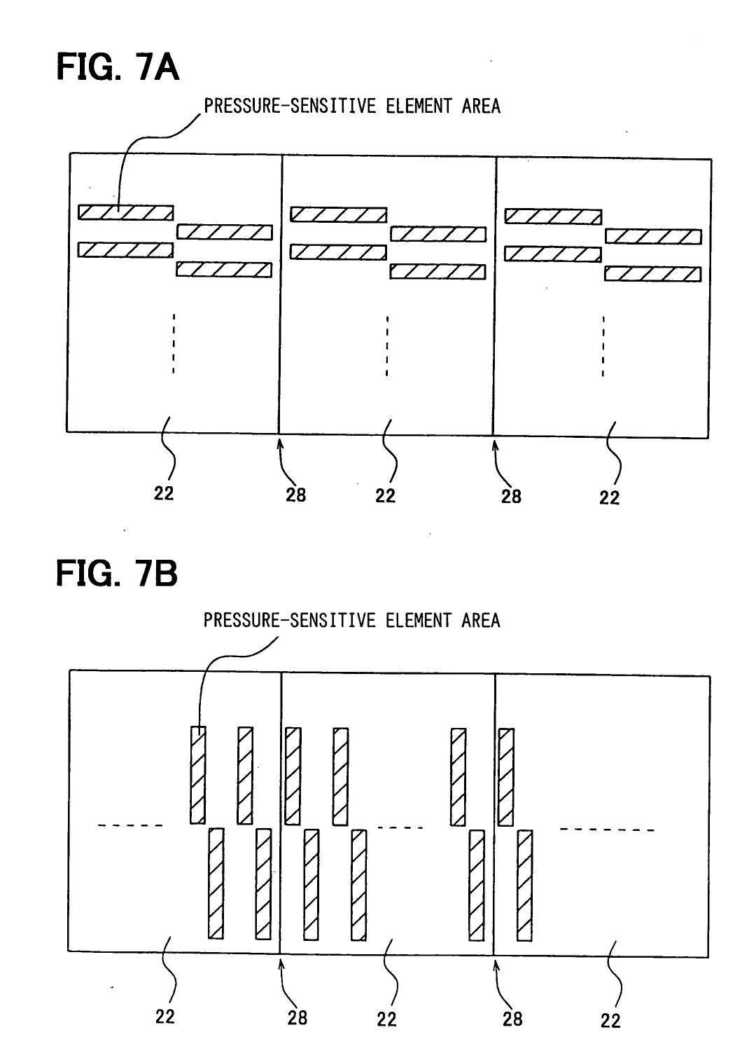

(b) In the above embodiment, the pressure-sensitive elements 221 are disposed in the staggered arrangement with a simple body unit of pressure-sensitive element 221 to thereby prevent the interference among the rubber pressure pieces 221a when folded. However, the same effect can be achieved by disposing the pressure-sensitive elements 221 in the staggered arrangement with an array unit as shown in FIGS. 7A-7B, for example. For example, in the case of FIG. 7A, the staggered arrangement is carried out for every array comprising a predetermined number of pressure-sensitive elements which are arranged in the lateral direction, thus in the direction perpendicular to the body height direction of a sleeper when the sensor sheet is put on a bed or the like. On the other hand, in the case of FIG. 7B, the staggered arrangement is carried out with an array unit comprising a predetermined number of pressure-sensitive elements which are arranged in the longitudinal direction, thus in the body height direction of a sleeper when the sensor sheet is put on a bed or the like. Even in these cases, the array-basis pressure-sensitive elements do not interfere with one another when the sheet is folded at the folding portions 28.

However, when the pressure-sensitive elements 221 are more uniformly arranged, it is preferable that the pressure-sensitive elements 221 are disposed in the staggered arrangement with a simple body unit of press-sensitive element 221 as described with reference to the above embodiment. It is expected that the uniform arrangement is more preferable from the view point of proper detection of living body information such as respiration information or the like.

(c) In the above embodiment, the pressure-sensitive elements 221 are used as an example of the sensing elements, however, sensing elements for sensing vibration in place of pressure may be used. Piezo film elements, PVDF elements or the like may be used as the vibration sensing elements.

The present invention is not limited to the above-described first embodiment, and various modifications may be made without departing from the spirit of the present invention.

1. A sensor sheet comprising a substantially rectangular sheet member and a plurality of sensing elements which are disposed in a predetermined distribution on the sheet member and sense pressure or vibration, the sensor sheet outputting a signal corresponding to load or vibration from a sleeper, wherein:
a lateral width of the sheet member is set to a minimum length at which the sensing elements exist in a predetermined range when the sleeper moves in a lateral direction; and
a longitudinal length of the sheet member is set to a minimum length at which the sensing elements exist in a predetermined range corresponding to a trunk part of the sleeper, and the sheet member is allowed to be folded at least in the lateral width direction.

2. The sensor sheet according to claim 1, wherein the longitudinal length of the sheet member is defined on the basis of an interval between tibial height and crotch height of the sleeper.

3. The sensor sheet according to claim 1, wherein the longitudinal length of the sheet member is defined on the basis of an interval between tibial height and sidebone of the sleeper.

4. The sensor sheet according to claim 1, wherein the longitudinal length of the sheet member is defined on the basis of length of a chest part in a body height direction of the sleeper.

5. The sensor sheet according to claim 1, wherein the sensing elements are designed so that transmission pieces disposed at one surface side of the sheet member so as to project from the one surface side transmit pressure or vibration to sensor portions disposed in the sheet member, and the sensing elements are disposed in a staggered arrangement so as to prevent the transmission pieces from abutting against one another when the sheet member is folded so that the transmission pieces face one another.

6. The sensor sheet according to claim 1, wherein the sheet member is equipped with a hard sheet portion for, when the sheet member is laid on bedding, reducing dispersion in downward displacement of the sensing elements by flexibility of the bedding, wherein the hard sheet portion is divided at the folding portion.

7. The sensor sheet according to claim 1, wherein the sheet member includes film-type protecting portions on upper and lower surfaces thereof, and the sensing elements are disposed between the film-type protecting portions to be isolated from external atmosphere.

8. The sensor sheet according to claim 1, wherein the film-type protecting portions are transparent.

9. A sensor sheet comprising a substantially rectangular sheet member and a plurality of sensing elements which are disposed in a predetermined distribution on the sheet member and sense pressure or vibration, the sensor sheet outputting a signal corresponding to load or vibration from a sleeper, wherein:
a lateral width of the sheet member is set to a minimum length at which the sensing elements exist in a predetermined range when the sleeper moves in a lateral direction;
a longitudinal length of the sheet member is set to a minimum length at which the sensing elements exist in a predetermined range corresponding to a trunk part of the sleeper, and the sheet member is allowed to be folded at least in the lateral width direction; and
the sheet member is equipped with a hard sheet portion for, when the sheet member is laid on bedding, reducing dispersion in downward displacement of the sensing elements by flexibility of the bedding, wherein the hard sheet portion is divided at the folding portion.

10. The sensor sheet according to claim 9, wherein the sheet member includes film-type protecting portions on upper and lower surfaces thereof, and the sensing elements are disposed between the film-type protecting portions to be isolated from external atmosphere.

11. The sensor sheet according to claim 10, wherein the sensing elements are designed so that transmission pieces disposed at one surface side of the sheet member so as to project from the one surface side transmit pressure or vibration to sensor portions disposed in the sheet member, and the sensing elements are disposed in a staggered arrangement so as to prevent the transmission pieces from abutting against one another when the sheet member is folded so that the transmission pieces face one another.
